(19) United States
(12) Patent Application Publication
(10) Pub. No.: US 2007/0243262 A1
(43) Pub. Date: Oct. 18, 2007

(54) Stable S-nitrosothiol formulations

(76) Inventors: Kevin P. Hurley, Chapel Hill, NC (US); Li Li Bovet, Chapel Hill, NC (US); Jonathan S. Stamler, Chapel Hill, NC (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/644,388

(22) Filed: Dec. 21, 2006

Related U.S. Application Data
(60) Provisional application No. 60/754,071, filed on Dec. 21, 2005.

(51) Int. Cl. A61K 38/06; A61K 9/14; C07K 5/037

(52) U.S. Cl. 424/499; 514/18; 530/331

(57) ABSTRACT

The invention provides stable S-nitrosothiol, such as S-nitrosoglutathione, formulations for long term storage and in vivo delivery of S-nitrosothiols. The invention provides stable aerosol formulations comprising S-nitrosothiol, such as S-nitrosoglutathione, and methods of treating patients in need of S-nitrosothiol, such as S-nitrosoglutathione, and/or nitric oxide treatment.

### RELATED APPLICATIONS

This application claims the benefit of U.S. Provisional Application No. 60/754,071, filed Dec. 21, 2005, the contents of which are incorporated herein by reference in its entirety.

### FIELD OF THE INVENTION

The present invention is directed to compositions and formulations comprising stabilized S-nitrosoglutathione and methods of using the same.

### BACKGROUND OF THE INVENTION

Nitric oxide (NO) is a ubiquitous molecule that has several biological functions, including decreasing blood pressure and inhibiting platelet function. To deliver NO bioactivity under physiological conditions, NO must be stabilized because it is too reactive by itself to reach a desired treatment location within the body. Current delivery methods typically involve polymers and small molecules, such as S-nitroso-D,L-penicillamine (SNAP) and S-nitrosocysteine (CysNO), that release NO in the body. These methods are flawed, however, because they release NO rapidly under physiological conditions and/or have a very short shelf life. Such methods are not able to deliver sufficient quantities of NO to a desired location for extended periods of time or in a controlled manner. Naturally occurring NO donor S-nitrosothiols (SNOs), such as S-nitrosoglutathione (GSNO) and S- nitrosocysteine, are particularly unstable. While both of these endogenous primary SNOs are more stable than tertiary SNOs thermodynamically, they are highly unstable kinetically at ambient temperatures and above. These issues make alternate technologies for delivery of S-nitrosothiols attractive, in particular as related to identifying methods for stabilizing S-nitrosothiols prior to delivery to patients while still allowing for spontaneous production of NO bioactivity under physiological conditions. Of particular value is identification of methods for kinetic stabilization (i.e., protection from redox and other reactions) of SNOs.

GSNO, a key endogenous source of NO bioactivity, has several biological functions that have generated clinical interest, particularly in cardiovascular and bronchopulmonary diseases and disorders. For example, GSNO has an inhibitory effect on platelet activation. GSNO also inhibits nuclear factor kappa-B (NF-κB) activation and smooth muscle cell proliferation. In addition, GSNO has certain cardioprotective effects, and has been shown to benefit patients following balloon angioplasty, as well as patients with acute myocardial infarction and unstable angina. GSNO can reduce the rate of cerebral embolization and has also been shown to induce apoptosis in T cells. In addition to providing benefits related to the cardiovascular system, GSNO is a powerful bronchodilator. GSNO has been demonstrated in vitro and ex vivo to reverse the airway epithelial molecular defect in cystic fibrosis, increasing the expression and function of the ΔF508 cystic fibrosis transmembrane regulator on epithelial cell surfaces. Also, endogenous GSNO levels are increased in the airway of patients having pneumonia and reduced in patients having cystic fibrosis or severe asthma.

While GSNO is an attractive compound for treating a variety of diseases, the compound itself is unstable, as described above, and is unstable in aqueous solutions, decomposing in hours. Therefore, there is a need for stable compositions and formulations of GSNO that can be stored for an adequate time and that are useful for delivery to patients in need of GSNO treatment and delivery of NO bioactivity to tissues.

### SUMMARY OF THE INVENTION

The invention provides compositions and formulations that stabilize S-nitrosothiols (SNOs), such as S-nitrosoglutathione (GSNO). The compositions and formulations enable long term storage and provide an effective means for delivering SNOs to a patient in need thereof.

The present invention provides a composition comprising S-nitrosoglutathione micronized into particles of about 1.5 μm to about 6.0 μm. The S-nitrosoglutathione can have a purity greater than 95.0% as determined by HPLC. The composition can contain less than 5.0% reduced and oxidized L-glutathione, less than 2.5% glutathione, less than 2.5% glutathione disulfide and/or less than 2.0% H₂O.

The present invention provides an S-nitrosoglutathione formulation comprising an S-nitrosoglutathione and a hydrofluorocarbon propellant. The S-nitrosoglutathione can be present in particles of about 1.5 μm to about 6.0 μm. The hydrofluorocarbon propellant can be HFA 134 or HFA 227. The formulation can further comprise one or more co-solvents. The co-solvent can be ethanol and it can be present in an amount of about 1% to about 20%. The formulation can further comprise one or more surfactants. The surfactant can be oleic acid, salts of oleic acid or oleyl alcohol. The surfactant can be present in an amount of about 1% to about 2% w/w with respect to the amount of S-nitrosoglutathione. The S-nitrosoglutathione in the formulation can be present in an amount of about 0.1 mg/actuation to about 2.0 mg/actuation. The S-nitrosoglutathione can be about 0.15 mg/actuation to about 1.5 mg/actuation. The S-nitrosoglutathione in the formulation can be administered in a unit dosage of about 0.1 mg/day to about 160.0 mg/day. The S-nitrosoglutathione is administered in an amount of about 1.5 mg/day to about 25 mg/day.

The present invention provides an S-nitrosoglutathione formulation comprising an S-nitrosoglutathione and a hydrofluorocarbon propellant, filled in a metal canister. The canister can have part or all of its internal metallic surfaces made of stainless steel, anodised aluminum lined with an inert organic coating, or anodised aluminum not lined with an inert organic coating. The inert organic coating can be epoxy-phenol resins, perfluoroalkoxyalkane, perfluoroalkoxyalkylene, perfluoroalkylenes such as polytetrafluoroethylene, fluorinated-ethylene-propylene, polyether sulfone and a copolymer fluorinated-ethylene-propylene polyether sulfone.

The present invention provides an S-nitrosoglutathione formulation comprising an S-nitrosoglutathione, HFA 134, 5% ethanol and 2% oleic acid.

The present invention provides an S-nitrosoglutathione formulation comprising an S-nitrosoglutathione micronized into particles of about 1.5 μm to about 6.0 μm, HFA 134, 5% ethanol and 2% oleic acid.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. Unless otherwise required by context, singular terms as used herein shall include pluralities and plural terms shall include the singular. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### DETAILED DESCRIPTION OF THE INVENTION

The present invention provides compositions and formulations that stabilize S-nitrosothiols (SNOs), such as S-nitrosoglutathione (GSNO). The compositions and formulations enable long term storage and provide an effective means for delivering SNOs to a patient in need thereof. As used herein, the term “S-nitrosothiols” includes, but is not limited to S-nitroso-beta-mercaptosuccinic acid, 1-S-nitrosothio-beta-D-galactopyranose, S-nitrosoglutathione (GSNO), S-nitroso-N-acetylcysteine (SNAC), S-nitrosothioglycerol, S-nitroso-N-acetylpenicillamine (SNAP), S-nitrosohomocysteine, S-nitrosocysteine (CysNO), S-nitrosocysteinylglycine. In an embodiment, the SNO is GSNO.

Particles

The present invention provides compositions and formulations in which the SNO is processed prior to inclusion in the compositions or formulations, in order to produce particles in the desired size range. For example, the SNO can be milled or micronized using suitable equipment for example an air jet mill, hammer mill, ball mill or using a microfluidizer. Alternatively, particles in the desired particle range may be obtained by, for example, spray drying or controlled crystallization methods, for example, crystallization using supercritical fluids or via an emulsion method, such as microfluidization or homogenization. Alternatively, SNO can be processed as described above during the formulation process, described in further detail below.

When the compositions or formulations of the invention are delivered to the lungs through an aerosol metered dose inhaler so as to produce a pharmacodynamic effect, the SNO particles can be about 0.5 μm to about 10 μm, about 1 μm to about 8 μm, or about 1 μm to about 5 μm (or any value within said range). In an embodiment, an SNO particle in a composition or formulation of the invention is about 1.5 μm to about 6 μm (or any value within said range). For some compositions and formulations about 90% of SNO particles in an SNO stabilizing formulation of the invention are less than about 6 μm, and about 50% are less than about 3 μm. In an embodiment, the SNO is GSNO.

The surfaces of the particles can also be modified prior to dispersion, for example, by spray drying a solution of drug and surfactant or by adsorption of surfactant onto SNO particles. Further techniques for modification of the surfaces of the particles can also be used, for example freeze drying, microfluidizing, and milling.

Hydrofluorocarbons

The present invention provides a formulation of the present invention comprising SNO and a hydrofluorocarbon (HFA) propellant. The HFA propellant can be 1,1,2-tetrafluoroethane (HFA-134), 1,1,1,2,3,3,3-heptafluoropropane (HFA-227), or a mixture of HFA-134 and HFA-227, for example a density matched mixture of HFA-134 and HFA-227. The amount of HFA propellant in a formulation can be about 80% w/w to about 98% w/w (or any value within said range). In an embodiment, the amount of HFA propellant is about 90% w/w to about 98% w/w (or any value within said range). For example, the amount of HFA propellant is about 91%, 92%, 93%, 94%, 95%, 96%, 97%, or 98% w/w.

Co-Solvents

The present invention provides a formulation comprising SNO and a hydrofluorocarbon (HFA) propellant and further comprising a co-solvent. The co-solvent can be ethanol. A small amount of ethanol (about 1-8% w/w, or about 1% to about 5% w/w (or any value within said range)), influences deposition characteristics of an aerosol drug, thereby improving systemic delivery, because ethanol is involved in reducing amounts of very small particles (0.5 μm -2 μm) which are normally exhaled after a short residence time in the lung. In addition, ethanol reduces the deposition of discharged materials on an inhaler actuator orifice. Therefore, dose reproducibility is improved after repeated administrations because the actuator orifice is kept clear of interfering materials. The amount of ethanol can be from 0% to about 20% w/w, from 0% to about 10% w/w, or from 0% to about 5% w/w (or any value within said range). In an embodiment, the amount of ethanol is about 5% w/w.

The co-solvent can also have a higher polarity than ethanol. The presence of a co-solvent having a higher polarity than ethanol allows reduction in the ethanol amount to allow the modulation of the particle size of the produced aerosol droplets. Co-solvents with a higher polarity than ethanol can be, for example, lower alkyl (C₁-C₄) alcohols, polyols, or polyalkylene glycols. Polyols include, but are not limited to, propylene glycol and glycerol. In an embodiment, the polyalkylene glycol is polyethylene glycol.

A formulation of the invention can comprise both ethanol and an additional co-solvent that has a higher polarity than ethanol, wherein the additional co-solvent is present in an amount from about 0. 1% to about 10% w/w, from about 0.2% to about 10% w/w, from about 0.5% to about 6% w/w, or from about 1% to about 2% w/w (or any value within said ranges).

Surfactants

The present invention provides a formulation comprising SNO, a hydrofluorocarbon (HFA) propellant, a co-solvent and further comprising a surfactant. The amount of surfactant that can be present in an SNO stabilizing formulation of the invention can range from about 0. 1% w/w to about 10% w/w (or any value within said range) with respect to the SNO. In an embodiment, the amount of surfactant present is at least 1% w/w with respect to the SNO. In an embodiment, the amount of surfactant present is up to about 5% w/w with respect to the SNO.

Examples of suitable surfactants include, but are not limited to, fatty acid, fatty acid esters including fatty acid trigylcerides, fatty alcohols, salts of fatty acids, oleyl alcohol, sorbitan mono-oleate, sorbitan monolaurate, polyoxyethylene (20) sorbitan monolaurate, polyoxyethylene (20) sorbitan mono-oleate, natural lecithin, oleyl polyoxyethylene (2) ether, stearyl polyoxyethylene (2) ether, lauryl polyoxyethylene (4) ether, block copolymers of oxyethylene and oxypropylene, oleic acid, salts of oleic acid, synthetic lecithin, diethylene glycol dioleate, tetrahydrofurfuryl oleate, ethyl oleate, isopropyl myristate, isopropyl palmitate, glyceryl mono-oleate, glyceryl monostearate, glyceryl monoricinoleate, cetyl alcohol, stearyl alcohol, cetyl pyridinium chloride, olive oil, glyceryl monolaurate, corn oil, cotton seed oil, sunflower seed oil, polyoxyethylenesorbitan monooleate, sorbitan trioleate, oligolactic acid, lecithin, (poly)alkoxy derivatives including polyalkoxy alcohols, in particular 2-(2-ethoxyethoxy) ethanol. Additional (poly)alkoxy derivatives include polyoxyalkyl ethers and esters, such as polyoxyethylene ethers and esters, including, but not limited to, polyoxyethylene alkyl ethers, polyoxyethylene sorbitan fatty acid esters and polyoxyethylene stearates. In an embodiment, the surfactant is oleic acid, salts of oleic acid or oleyl alcohol. The oleic acid, salts of oleic acid or oleyl alcohol can be present at about 2% w/w with respect to the SNO. A composition or formulation of the invention can optionally comprise additional ingredients, such as additives that serve as preservatives, antioxidants, radical quenchers, sweeteners, taste masking agents, pharmaceutically active agents, adjuvants, carriers, buffers, chemical stabilizers, and/or polymers. The amount of additional ingredients included in a formulation of the invention can be, for example, 0% to about 1% w/w (or any value within said range).

Impurities

The present invention provides compositions and formulations which contain limited impurities. The compounds and formulations of the present invention have a purity greater than or equal to about 95.0% as determined by known methods in the art, for example, HPLC. In an embodiment, the compounds and formulations of the present invention have a purity ranging from about 95.0% to about 100% (or any value within said range).

In order to elicit the maximum pharmacodynamic and therapeutic effect of the compositions and formulations of the present invention, it is beneficial to limit the levels of reduced and oxidized L-glutathione impurities. These impurities can result in undesirable toxicity. The compounds and formulations of the present invention contain less than about 5.0% reduced and oxidized L-glutathione. In an embodiment, the compounds and formulations of the present invention contain reduced and oxidized L-glutathione in a range from about 0.0% to about 5.0% (or any value within said range). It is beneficial to limit the levels of glutathione (GSH) and glutathione disulfide (GSSG) present in the compositions and formulations; thus, the compounds and formulations of the present invention contain less than about 2.0%-2.5% glutathione and less than about 2.0% - 2.5% glutathione disulfide. In an embodiment, the compounds and formulations of the present invention contain glutathione and glutathione disulfide in a range from about 0.0% to about 2.5% (or any value within said range), respectively. It is also beneficial to limit the amount of H₂O present within the composition or formulation; thus, the compounds and formulations of the present invention contain less than about 2.0% H₂O. In an embodiment, the compounds and formulations of the present invention contain H₂O in a range from about 0.0% to about 2.0% (or any value within said range).

Disorders

The present invention also provides methods of treating a subject afflicted with a disorder ameliorated by NO donor therapy (i.e., conditions or disorders where SNO treatment is desirable) where the method comprises administering to the subject a therapeutically effective amount of the compositions and formulations as defined above, or a pharmaceutically acceptable salt thereof, or a prodrug or metabolite thereof, in combination with a pharmaceutically acceptable carrier. The subject can be e.g., any mammal, e.g., a human, a primate, mouse, rat, dog, cat, cow, horse, pig. For example, the mammal is a human.

As used herein the term “therapeutically effective amount” means the amount necessary to alleviate at least one symptom of a disorder to be treated as described herein. In an embodiment, the therapeutically effective amount is any amount of SNO delivered by single or multiple actuations of an inhaler able to produce a pharmacodynamic effect.

As used herein, “treating” describes the management and care of a patient for the purpose of combating a disease, condition, or disorder and includes the administration of a compound of the present invention to prevent the onset of the symptoms or complications, alleviating the symptoms or complications, or eliminating the disease, condition or disorder. More specifically, treating includes reversing, attenuating, alleviating, minimizing, suppressing or halting at least one deleterious symptom or effect of a disease (disorder) state, disease progression, disease causative agent (e.g., bacteria or viruses), or other abnormal condition. Treatment is continued as long as symptoms and/or pathology ameliorate.

The disease, conditions or disorders can include, but are not limited to, cystic fibrosis, asthma, and other pulmonary disorders involving diminished gas exchange or inflammation such as pulmonary fibrosis, and pneumonia, cardiovascular proliferative, inflammatory, contractile and hypertensive disorders, including hypertension, atherosclerosis, restenosis, ischemia and heart failure; preconditioning related disorders of the heart and brain; motility and smooth muscle disorders of the GI tract, including esophageal spasm, biliary spasm, and colic; erectile dysfunction stroke; infectious disease (viral, bacterial and other), disorders of red blood cells characterized by SNO deficiency, abnormal rheology or impaired vasodilation, such as sickle cell disease and stored blood-related diathesis, and thrombotic disorders.

Pharmaceutical Compositions/Formulations

A pharmaceutical composition is a formulation containing the disclosed compounds in a form suitable for administration to a subject. A pharmaceutical composition of the invention is preferably formulated to be compatible with its intended route of administration. Examples of routes of administration include oral and parenteral, e.g., intravenous, intradermal, subcutaneous, inhalation, transdermal (topical), transmucosal, and rectal administration. Solutions or suspensions can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic. In an embodiment, the compositions and formulations of the present invention are administered as an aerosol for administration by inhalation. The compounds are delivered in the form of an aerosol spray from pressured container or dispenser that contains a suitable propellant, e.g., a gas such as carbon dioxide, HFA or a nebulizer.

The active reagents can be prepared with carriers that will protect against rapid elimination from the body. For example, a controlled release formulation can be used, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

The compositions and formulations of the instant invention can also comprise one or more desiccants. Suitable desiccants that can be used in the present invention are those that are pharmaceutically safe, and include, for example, pharmaceutical grades of silica gel, crystalline sodium, potassium or calcium aluminosilicate, colloidal silica, anhydrous calcium sulphate and the like. The desiccant may be present in an amount from about 1.0% to 20.0%, or from about 2% to 15% w/w (or any value within said range).

The present invention provides compositions and formulations comprising SNO where the concentration of SNO present within the composition or formulation is at least about 0.01% w/w (as used herein, w/w refers to weight of a component with respect to the total can fill weight, i.e. the weight of the total contents of the can filled with all components as described herein), preferably at least about 0.05% w/w, more preferably between about 0. 1% w/w and about 1.0% w/w, even more preferably at least about 1.0% w/w. SNO can be dissolved or dispersed in the propellant, co-solvent and/or surfactant as described above. In an embodiment, the SNO is GSNO.

The present invention provides compositions and formulations suitable for delivering a therapeutic amount of the SNO to the lungs of a patient in need thereof via a pressurized metered dose inhaler (PMDI) in about 1 to about 200 actuations/day (or any value within said range) by a metering valve capable of delivering about 25 μl to about 200 Al (or any value within said range). In an embodiment, the composition and formulation is delivered in about 1 to about 4 actuations/day (or any value within said range). In an embodiment, the metering valve is capable of delivering about 50 μl to about 100 μl (or any value within said range). Advantageously the formulation will be suitable for delivering a therapeutic dose of at least about 0.1 mg/actuation to about 2.0 mg/actuation (or any value within said range).

It is especially advantageous to formulate compositions in dosage unit form for ease of administration and uniformity of dosage. Dosage unit form as used herein refers to physically discrete units suited as unitary dosages for the subject to be treated; each unit containing a predetermined quantity of active reagent calculated to produce the desired therapeutic effect in association with the required pharmaceutical carrier. The specification for the dosage unit forms of the invention are dictated by and directly dependent on the unique characteristics of the active reagent and the particular therapeutic effect to be achieved, and the limitations inherent in the art of compounding such an active agent for the treatment of individuals.

When the SNO comprised within the formulation is GSNO, the formulation comprises about 0.1 mg/actuation to about 2.0 mg/actuation (or any value within said range). In an embodiment, the formulation comprises about 0.15 mg/actuation to about 1.5 mg/actuation (or any value within said range). The unit dosage of a formulation comprising SNO can be about 0.1 mg/day to about 160 mg/day (or any value within said range). In an embodiment, the formulation can be about 1.5 mg/day to about 25 mg/day (or any value within said range).

Suspension (or dispersion) formulations of the SNO can be prepared using one of three basic approaches: cold filling, two stage filling, and single stage filling. For cold filling the SNO (API), propellant, co-solvent and any other excipients can be mixed and homogenized in a low temperature vessel (typically −50° C.) and re-circulated through the equipment metering head. A volume of the formulation can be metered into the open canister at low temperature and then the valve can be quickly placed and crimped. When the canister returns to room temperature the pressure inside the canister rises to its intended value.

Alternately, a two stage filling process can be used. For example, the API and excipients can be either dissolved or dispersed in the co-solvent, and then this mixture can be accurately metered by volume into the open container. The valve can be placed and crimped to the canister and then the propellant is forced into the canister through the valve.

Alternately, a single stage pressure filling process can be used. For example, the API, propellant, co-solvent and any other excipients can be mixed and homogenized in a pressurized mixing vessel and recirculated through the equipment metering head. The valve can be crimped to the canister, often with some form of purging (exclusion of the air). A precise volume of liquid containing the mixture is forced into the canister through the valve.

Suspension (or dispersion) formulations can also be prepared by co-preparation of particles with excipients, for example by spray-drying, to form composite particles. Solution formulations of the invention can be prepared by adding API, co-solvent and any other excipients to the HFA propellant by pressure filling or cold filling methods.

PMDI Components

The formulations of the invention can also be filled into canisters (also referred to herein as “cans”) suitable for delivering pharmaceutical aerosol formulations. Aerosol canisters for use with the formulations of the invention can comprise a valve and actuator for delivery to a patient for the treatment of diseases and/or conditions that would benefit from in vivo delivery of SNO and/or nitric oxide to specific tissue sites.

The canister can be a metal can, for example, an aluminum can, closed with a metering valve. Cans that are suitable for use according to the methods of the invention can be obtained, for example, from Presspart T&M (Watertown, Conn.) and 3M Neotechnic Ltd (UK). In an embodiment, formulations can be filled into cans having part or all of the internal surfaces made of anodised aluminum, stainless steel, or lined with an inert organic coating. Examples of preferred coatings include, but are not limited to, epoxy-phenol resins, perfluoroalkoxyalkane, perfluoroalkoxy alkylene, perfluoroalkylenes such as polytetrafluoro-ethylene, fluorinated-ethylene-propylene, polyether sulfone and a copolymer fluorinated-ethylene-propylene polyether sulfone. Other suitable coatings could be polyamide, polyimide, polyamideimide, polyphenylene sulfide or their combinations. Cans suitable for comprising a formulation of the invention can have a rolled-in rim, or a part or full rollover rim.

A metering valve and type of gasket can be chosen according to the knowledge of those skilled in the art. The gasket may comprise any suitable elastomeric material such as low density polyethylene, ethylene propylene diene monomer (EPDM), chloroprene, butyl (i.e. bromobutyl), and thermoplastic elastomers (TPE). Non-limiting examples of suitable valves include those that are commercially available from manufacturers well known in the aerosol industry, such as Valois (France), Bespak Europe (King's Lynn, UK), and 3M Neotechnic Ltd (UK). In additional embodiments, the internal surfaces of metal valve components in contact with the formulation of the invention can be coated with an inert material.

Valve actuators can comprise orifice diameter from about 0.10 mm to about 0.50 mm (or any value within said range) can be generally used with the aerosol formulations of the invention. In an embodiment, the orifice diameter can be about 0.12 mm, 0.14 mm, 0.16 mm, 0.18 mm, 0.22 mm, 0.33 mm, 0.42 mm or 0.48 mm).

The following examples, including the experiments conducted and results achieved are provided for illustrative purposes only and are not to be construed as limiting the invention.

### EXAMPLES

GSNO Stability Studies

The capability of HFA to stabilize S-nitrosoglutathione (GSNO) was investigated by producing pMDI formulations at various percentages of ethanol in the propellant HFA-134a (Ineos Fluor, St. Gabriel, La.).

For 0.15 mg/Actuation and a Total of 120 Actuations Per Canister:

A two-stage filling process was used to prepare GSNO canisters. In stage one, 0.0180 g of GSNO (Chemical Synthesis Services, Craigavon, Co. Armagh, Northern Ireland) was placed into an open 3M Neotechnic 19-mL can, which was internally coated (IntraPac Inc., Harrisonburg, Va.) with fluorinated ethylene propylene and polyether sulfone (FEP/PES). After addition of GSNO, 0.1436 g (1% w/w based on propellant), 0.6906 g (5% w/w based on propellant), or 1.3183 g (10% w/w based on propellant) of ethanol was added to the can with 0.0004 g oleic acid (2% w/w based on active). In stage 2, a BK 357 valve (Bespak Europe, King's Lynn, UK) with a 100-μL metering chamber was crimped onto the can and propellant HFA-134a (Ineos Fluor, St. Gabriel, La.) was dispensed through the valve into the can. Cans were filled at ambient room temperature. Each canister was fitted with Bespak actuators (Bespak Europe, King's Lynn, UK) having 0.48 mm orifice diameter.

For 1.50 mg/Actuation and a Total of 120 Actuations Per Canister:

A two-stage filling process was used to prepare GSNO inhalers. In stage one, 0.1800 g of GSNO was placed into an open 3M Neotechnic 19-mL can, which was internally coated with fluorinated ethylene propylene and polyether sulfone (FEP/PES). After addition of GSNO, 0.1419 g (1% w/w based on propellant), 0.6837 g (5% w/w based on propellant), or 1.3033 g (10% w/w based on propellant) of ethanol was added to the can with 0.0036 g oleic acid (2% w/w based on active). In stage 2, a BK 357 valve (Bespak Europe, King's Lynn, UK) with a 100-μL metering chamber was crimped onto the can and propellant HFA-134a (Ineos Fluor, St. Gabriel, La.) was dispensed through the valve into the can. Cans were filled at ambient room temperature. Each canister was fitted with Bespak actuators (Bespak Europe, King's Lynn, UK) having 0.48 mm orifice diameter.

GSNO powder was tested for stability at various temperatures over a three-month period of time, and each formulation was tested for stability at various temperatures over a six month period of time. Stability was determined by HPLC analysis at various times throughout the test period. The results are shown in Tables 1-4.

Table 1 shows the stability of GSNO powder at 5° C., −20° C., and −80° C. over a three month period.

TABLE 1
GSNO API Stability

| Elapsed | GSNO % w/w | GSNO % w/w | GSNO % w/w |
|---|---|---|---|
| Days | at 5° C. | at −20° C. | at −80° C. |
| 0 | 92.7 | 92.7 | 92.7 |
| 14 | 84.6 | 90.6 | — |
| 30 | 89.2 | 91.1 | 91.4 |
| 90 | 80.0 | 89.7 | 91.9 |

As shown, GSNO is more stable at very cold temperatures (such as −20° C. and −80° C.), and it rapidly degrades at temperatures above 0° C.

Table shows the results of formulations comprising 1% ethanol stored at cold (5° C.), e (25° C.), and accelerated storage (40° C. at 75% relative humidity).

TABLE 2
GSNO API stability in HFA formulation containing 1% Ethanol

| Elapsed | GSNO % w/w | GSNO % w/w | GSNO % w/w at |
|---|---|---|---|
| Days | at 5° C. | at 25° C. | 40° C./75% RH |
| 0 | 95.5 | 95.5 | 95.5 |
| 14 | 93.6 | 93.4 | 90.2 |
| 30 | 91.5 | 92.9 | 86.1 |
| 90 | 91.6 | 90.3 | 77.0 |
| 180 | 93.7 | 77.0 | 72.7 |

Table 3 shows the results of formulations comprising 5% ethanol stored at cold (5° C.), ambient temperature (25° C.), and accelerated storage (40° C. at 75% relative humidity).

TABLE 3
GSNO API stability in HFA formulation containing 5% Ethanol

| Elapsed | GSNO % w/w | GSNO % w/w | GSNO % w/w at |
|---|---|---|---|
| Days | at 5° C. | at 25° C. | 40° C./75% RH |
| 0 | 95.4 | 95.4 | 95.4 |
| 14 | 91.9 | 93.4 | 90.3 |
| 30 | 92.7 | 92.4 | 87.3 |
| 90 | 95.0 | 97.5 | 71.6 |
| 180 | 94.4 | 91.9 | 72.3 |

Table 4 shows the results of formulations comprising 10% ethanol stored at cold (5° C.), ambient temperature (25° C.), and accelerated storage (40° C. at 75% relative humidity).

TABLE 4
GSNO API stability in HFA formulation containing 10% Ethanol

| Elapsed | GSNO % w/w | GSNO % w/w | GSNO % w/w at |
|---|---|---|---|
| Days | at 5° C. | at 25° C. | 40° C./75% RH |
| 0 | 94.3 | 94.3 | 94.3 |
| 14 | 92.2 | 92.1 | 86.6 |
| 30 | 93.1 | 92.3 | 84.6 |
| 90 | 92.7 | 88.8 | 70.9 |
| 180 | 94.1 | 88.7 | 70.4 |

The results of the studies summarized in Tables 2-4 show that the stability of GSNO is greatly enhanced when formulated in HFA as described herein when compared to the unformulated powder. This allows for the successful manufacture and storage at ambient and low temperatures of HFA-based formulations of GSNO. These data show that the propellant HFA increased the stability of GSNO, especially at lower temperatures (i.e., 4-5° C. or −20° C.) more than at higher temperatures (i.e., room temperature or higher) thereby enabling its successful storage in HFA at ambient and cold temperatures.

It should be understood that the foregoing disclosure emphasizes certain specific embodiments of the invention and that all modifications or alternatives equivalent thereto are within the spirit and scope of the invention as set forth in the appended claims.

What is claimed is:

1. A composition comprising S-nitrosoglutathione micronized into particles of about 1.5 μm to about 6.0 μm.

2. The composition of claim 1 wherein the S-nitrosoglutathione has a purity greater than 95.0% as determined by HPLC.

3. The composition of claim 3, wherein the composition contains less than 5.0% reduced and oxidized L-glutathione.

4. The composition of claim 3, wherein the composition contains less than 2.5% glutathione.

5. The composition of claim 3, wherein the composition contains less than 2.5% glutathione disulfide.

6. The composition of claim 3, wherein the composition contains less than 2.0% H₂O.

7. An S-nitrosoglutathione formulation comprising an S-nitrosoglutathione and a hydrofluorocarbon propellant.

8. The formulation of claim 7, wherein the S-nitrosoglutathione is present in particles of about 1.5 μm to about 6.0 μm.

9. The formulation of claim 7, wherein the hydrofluorocarbon propellant is HFA 134 or HFA 227.

10. The formulation of claim 7, further comprising one or more co-solvents.

11. The formulation of claim 10, wherein the co-solvent is ethanol.

12. The formulation of claim 11, wherein the ethanol is present in an amount of about 1% to about 20%.

13. The formulation of claim 7, further comprising a surfactant.

14. The formulation of claim 13, wherein the surfactant is oleic acid, salts of oleic acid or oleyl alcohol.

15. The formulation of claim 13, wherein the surfactant is present in an amount of about 1% to about 2% w/w with respect to the amount of S-nitrosoglutathione.

16. The formulation of claim 7, wherein the S-nitrosoglutathione is present in an amount of about 0.1 mg/actuation to about 2.0 mg/actuation.

17. The formulation of claim 16, wherein the S-nitrosoglutathione is present in an amount of about 0.15 mg/actuation to about 1.5 mg/actuation.

18. The formulation of claim 7, wherein the S-nitrosoglutathione is administered in a unit dosage of about 0.1 mg/day to about 160.0 mg/day.

19. The formulation of claim 18, wherein the S-nitrosoglutathione is administered in an amount of about 1.5 mg/day to about 25 mg/day.

20. The formulation of claim 7, filled in a metal canister.

21. The formulation of claim 20, wherein the canister has part or all of its internal metallic surfaces made of stainless steel, anodised aluminum lined with an inert organic coating, or anodised aluminum not lined with an inert organic coating.

22. The formulation of claim 21, wherein the inert organic coating selected from epoxy-phenol resins, perfluoroalkoxyalkane, perfluoroalkoxyalkylene, perfluoroalkylenes such as polytetrafluoroethylene, fluorinated-ethylene-propylene, polyether sulfone and a copolymer fluorinated-ethylene-propylene polyether sulfone.

23. An S-nitrosoglutathione formulation comprising an S-nitrosoglutathione, HFA 134, 5% ethanol and 2% oleic acid.

24. An S-nitrosoglutathione formulation comprising an S-nitrosoglutathione micronized into particles of about 1.5 μm to about 6.0 μm, HFA 134, 5% ethanol and 2% oleic acid.
